(19) United States
(12) Patent Application Publication
(10) Pub. No.: US 2018/0258459 A1
(43) Pub. Date: Sep. 13, 2018

(54) NITRATE BIOSENSOR

(71) Applicant: William Marsh Rice University, Houston, TX (US)

(72) Inventors: Jeffrey J. TABOR, Houston, TX (US); Brian LANDRY, Houston, TX (US); Nikola DYULGYAROV, Houston, TX (US)

(21) Appl. No.: 15/760,957

(22) PCT Filed: Sep. 16, 2016
(86) PCT No.: PCT/US16/52281

§ 371 Date: Mar. 16, 2018

Related U.S. Application Data
(60) Provisional application No. 62/220,118, filed on Sep. 17, 2015.

(51) Int. Cl. C12Q 1/12; C12Q 1/6897; C12N 9/12; C12N 9/02

(52) U.S. Cl. CPC C12Q 1/12; C12Q 1/6897; C07K 2319/80; C12Y 207/13003; C12N 9/0004; C12N 9/12

(57) ABSTRACT

A nitrate sensing biosensor and bacteria and applications relating to the use of same are described. The nitrate biosensor comprises a two component sensor system (TCS) comprising: a nitrate-sensing sensor kinase (SK) gene comprising a ligand binding domain operably coupled to a kinase domain, and, a cognate response regulator (RR) gene comprising a receiver domain operably coupled to an DNA binding domain (DBD), as well as an output promoter that binds said DBD that is operably coupled to a heterologous reporter gene.

### PRIOR RELATED APPLICATIONS

This application claims priority to U.S. Ser. No. 62/220,118, entitled NITRATE BIOSENSOR FOR DETECTION OF GUT INFLAMMATION, filed Sep. 17, 2015, and incorporated by reference herein in its entirety for all purposes.

### FEDERALLY SPONSORED RESEARCH STATEMENT

This invention was made with government support under N00014-14-1-0487 awarded by the Office of Naval Research. The government has certain rights in the invention.

### FIELD OF THE DISCLOSURE

The invention includes novel materials, methods and systems using a two-component sensor kinase system for detecting nitrate.

### BACKGROUND OF THE DISCLOSURE

A two-component regulatory system serves as a basic stimulus-response coupling mechanism to allow organisms to sense and respond to changes in environmental conditions. Such systems typically consist of a membrane-bound histidine kinase that senses a specific environmental stimulus and a corresponding response regulator that mediates the cellular response, mostly through differential expression of target genes.

Signal transduction occurs through the transfer of phosphoryl groups from adenosine triphosphate (ATP) to a specific histidine residue in the histidine kinases (HK). This is an autophosphorylation reaction. The response regulators (RRs) were shown to be phosphorylated on an aspartate residue and to be protein phosphatases for the histidine kinases. The response regulators are therefore enzymes with a covalent intermediate that alters response-regulator output function. Phosphorylation causes the response regulator's conformation to change, usually activating an attached output domain, which then leads to the stimulation (or repression) of expression of target genes. The level of phosphorylation of the response regulator controls its activity. Some HKs are bifunctional, catalyzing both the phosphorylation and dephosphorylation of their cognate RR. The input stimuli can regulate either the kinase or phosphatase activity of the bifunctional HK. See e.g., FIG. 1.

Two-component signal transduction systems thus enable bacteria to sense, respond, and adapt to a wide range of environments, stressors, and growth conditions. These systems have been adapted to respond to a wide variety of stimuli, including nutrients, cellular redox state, changes in osmolarity, quorum signals, antibiotics, temperature, chemoattractants, pH and more. Some bacteria can contain up to as many as 200 two-component sensor systems that need tight regulation to prevent unwanted cross-talk.

In Escherichia coli, for example, the EnvZ/OmpR osmoregulation system controls the differential expression of the outer membrane porin proteins OmpF and OmpC. The KdpD sensor kinase proteins regulate the kdpFABC operon responsible for potassium transport in bacteria including E. coli and Clostridium acetobutylicum. The N-terminal domain of this protein forms part of the cytoplasmic region of the protein, which may be the sensor domain responsible for sensing turgor pressure.

A variant of the two-component system is the phospho-relay system. See FIG. 1, lower panel. Here a hybrid HK autophosphorylates and then transfers the phosphoryl group to an internal receiver domain, rather than to a separate RR protein. The phosphoryl group is then shuttled to histidine phosphotransferase (HPT) and subsequently to a terminal RR, which can evoke the desired response.

Signal transducing histidine kinases are the key elements in two-component signal transduction systems. Examples of histidine kinases are EnvZ, which plays a central role in osmoregulation, and CheA, which plays a central role in the chemotaxis system. Histidine kinases usually have an N-terminal ligand-binding domain and a C-terminal kinase domain, but other domains may also be present. The kinase domain is responsible for the autophosphorylation of the histidine with ATP, the phosphotransfer from the kinase to an aspartate of the response regulator, and (with bifunctional enzymes) the phosphotransfer from aspartyl phosphate back to ADP or to water. The kinase core has a unique fold, distinct from that of the Ser/Thr/Tyr kinase superfamily.

HKs can be roughly divided into two classes: orthodox and hybrid kinases. Most orthodox HKs, typified by the E. coli EnvZ protein, function as periplasmic membrane receptors and have a signal peptide and transmembrane segment(s) that separate the protein into a periplasmic N-terminal sensing domain and a highly conserved cytoplasmic C-terminal kinase core. Members of this family, however, have an integral membrane sensor domain. Not all orthodox kinases are membrane bound, e.g., the nitrogen regulatory kinase NtrB (GlnL) is a soluble cytoplasmic HK.

Hybrid kinases contain multiple phosphodonor and phosphoacceptor sites and use multi-step phospho-relay schemes instead of promoting a single phosphoryl transfer. In addition to the sensor domain and kinase core, they contain a CheY-like receiver domain and a His-containing phosphotransfer (HPt) domain.

It is possible to identify TCSs from bacterial genome sequences by computational methods, such as homology and/or domain searching. However, such TCSs typically sense unknown inputs and control unknown output genes. Because both key pieces of information are lacking, and the microbes that contain them are often un-culturable or difficult to genetically manipulate in the laboratory, making it very difficult to identify the inputs that they sense. Therefore, while TCSs have tremendous medical, industrial and basic research applications, they have not yet fully been exploited.

This application explores the use of particular TCSs for use in detecting nitrate, as well as disease such as gut inflammation or infection, but other uses are also described herein.

The gut nurtures growth of fermentative anaerobes (such as Clostridia, Bacteroidia, and the like, see FIG. 2, that convert complex polysaccharides into simple end products that the host uses for energy and strengthen the intestinal barrier. The various toxins produced by minor gut members (e.g. H₂S) can trigger an inflammatory response, resulting in reactive oxygen (e.g. O₂⁻) and nitrogen species (e.g. NO) that generate oxidized compounds such as nitrate (NO₃⁻) and tetrathionate (S₄O₆²⁻).

Rare facultative anaerobes can respire oxidized compounds, resulting in dysbiosis—an unhealthy change in the normal bacterial ecology of e.g., the intestines or the oral cavity. Dysbiosis then results in a weakened intestinal barrier, which permits bacterial toxins (e.g. lipopolysaccharide) to transit to bloodstream, where they can lead to metabolic syndrome, obesity, anxiety, and other symptoms.

The biological nitrogen cycle involves step-wise reduction of nitrogen oxides to ammonium salts and oxidation of ammonia back to nitrites and nitrates by plants and bacteria. Neither process was thought to have relevance to mammalian physiology. However in recent years, the salivary bacterial reduction of nitrate to nitrite has been recognized as an important metabolic conversion in humans.

Several enteric bacteria have also shown the ability of catalytic reduction of nitrate to ammonia via nitrite during dissimilatory respiration. However, the importance of this pathway in bacterial species colonizing the human intestine has been little studied. Researchers have found that the presence of 5 mM nitrate provided a growth benefit and induced both nitrite and ammonia generation in E. coli and L. plantarum bacteria grown at oxygen concentrations compatible with the content in the gastrointestinal tract. Nitrite and ammonia accumulated in the growth medium when at least 2.5 mM nitrate was present. Time-course curves suggest that nitrate is first converted to nitrite and subsequently to ammonia.

Strains of L. rhamnosus, L. acidophilus and B. longum infantis grown with nitrate produced minor changes in nitrite or ammonia levels in the cultures. However, when supplied with exogenous nitrite, NO gas was readily produced independently of added nitrate. Bacterial production of lactic acid causes medium acidification that in turn generates NO by non-enzymatic nitrite reduction. In contrast, nitrite was converted to NO by E. coli cultures even at neutral pH. It is thus believed that the bacterial nitrate reduction to ammonia, as well as the related NO formation in the gut, could be an important aspect of the overall mammalian nitrate/nitrite/NO metabolism and is yet another way in which the microbiome links diet and health.

Like Salmonella, pathogenic E. coli, including EPEC, EHEC, and C. rodentium, may benefit from intestinal inflammation. In the inflamed intestine, intestinal epithelium and recruited neutrophils and macrophages that express inducible nitric oxide synthetase (iNOS), upregulate the production of nitrate (NO³⁻). Obligate anaerobes, such as Bacteroidetes or Firmicutes that are the vast majority of healthy microbial community in the gut, cannot utilize nitrate as an electron acceptor. Rather, nitrate reductase-harboring facultative anaerobes, such as E. coli, can utilize NO³⁻ to generate energy for growth, leading to a growth advantage over obligate anaerobes in the inflamed intestine. Although this mechanism of E. coli overgrowth within the inflamed gut involves commensal-commensal competition, pathogenic E. coli strains, which bear nitrate reductase genes, such as narZ, in their genome, may use a similar mechanism to acquire a growth advantage over the competitive commensal community. Furthermore, the host inflammatory environment can act as a signal to trigger and enhance virulence factor expression. Thus, pathogens can take advantage of the inflammatory response to promote their growth in host tissues.

This advantage in inflammatory conditions can lead to major blooms of enterobacteria and a dramatic alteration of the gut microbial population or dysbiosis. Therefore, an ability to detect and measure physiological concentrations of inflammatory indicators, such as nitrate, at the site of inflammation would provide a novel measure of gut health. A bacterial sensor is an ideal solution because it can pass through the gut as a non-invasive observer while providing a readout of gut health that does not require removal of human tissue. Also, many usable bacterial strains are already approved for human consumption (probiotics). As an alternative, the gut flora can be sampled and applied to the biosensors in a table top ex vivo experiment, instead of performing the assay in vivo. Purified proteins could also be used in an in vitro experiment.

Related two-component systems can be mined using bioinformatics, characterized, and incorporated into a gut-friendly host for diagnostic use. We have identified several uncharacterized two-component system sensors in Shewanella species and related organisms that are predicted to sense a variety of terminal electron acceptors known to be markers of inflammation. Shewanella species demonstrate remarkable versatility in their ability to couple reduction of terminal electron acceptors to energy production and do so using an enhanced collection of reductases and associated transcriptional regulators to fine-tune their metabolic capabilities. They are therefore ideal candidates for sensor mining because these sensors likely demonstrate strong substrate specificity and respond to a broad range of ligand concentrations that correspond to their diverse environmental niches. These sensors could be combined with the nitrate and other existing sensors developed by our lab for implementation in non-invasive diagnostics.

### SUMMARY OF THE DISCLOSURE

This invention relates to nitrate biosensors made of a nitrate-sensing SK and its cognate RR, which can be rewired if needed for compatibility in the host organism or to increase the output signal. These two proteins are combined with an output promoter, responsive to the RR or rewired RR, where that output promoter is operably coupled to a reporter gene for diagnostic uses, or to a gene encoding a therapeutic protein for therapeutic uses.

In our proof of concept work, we first transported the nitrate binding and signaling protein NarX (UniProt P0AFA2) from its native host Escherichia coli to the probiotic host Bacillus subtilis.

NarX sits in the membrane of bacterial cells and binds extracellular nitrate. Upon binding nitrate, NarX undergoes a shape change, which alters its phosphorylation based signaling activity and allows it to activate its cognate RR-NarL or NarQ.

To transport the NarX signal from the membrane to the genome of B. subtilis, we created an engineered protein (NarL/YdfI) composed of the first half of the E. coli NarL (UniProt P0AF28) protein and the second half of the B. subtilis YdfI protein (GenBank BAA19376.1). This rewired RR is capable of receiving the phosphorylation-based signal from the NarX protein. This was required because previous attempts at transferring the natural NarL protein with its associated promoter failed to result in functional transcription in B. subtilis.

When the hybrid NarL/YdfI receives the signal from the activate SK, it in turn interacts with the third element in our system, the native YdfJ promoter (P_{YdfJ}) in B. subtilis, which has been operatively coupled to another gene, thus changing its expression. P_{YdfJ} is composed of a DNA sequence that can interact with the engineered NarL/YdfI protein, and upon interaction, stimulate production of an arbitrary RNA transcript, which can encode reporter proteins, such as GFP, enabling measurement of nitrate concentration, or therapeutic proteins such as those that make Polymyxin B, an antibiotic.

Thus, by genetic engineering we are enabling the creation of smart bacteria, which can be administered as therapeutic agents. These bacteria are capable of diagnosing the disease state of the patient inside their own body and upon diagnosis the bacteria produce relevant therapeutic molecule to treat the disease. A key target of these treatments are autoimmune diseases such as arthritis, diabetes, and irritable bowel syndrome, in which up-regulation of immune signaling leads to the production of nitric oxide and its oxidized form nitrate. We have created a novel protein based nitrate sensing system and demonstrated its functionality in bacteria. This will enable smart bacteria to diagnose autoimmune diseases based on the concentration of nitrate present within human gut, enabling non-invasive diagnosis and treatment of a wide variety of highly prevalent diseases.

Some major potential uses are:

1. To create novel therapeutic bacteria, which are capable of sensing nitrate in the gut, to diagnose diabetes and response by treatment with polymyxin B, a molecule that has been shown to ameliorate diabetes symptoms by eliminating the causative bacterial produced chemicals.

2. To create novel therapeutic bacteria, which are capable of sensing nitrate in the gut, to diagnose irritable bowel syndrome and respond by producing Lactobacillus rhamnosus GG protein p40 which has been shown to activate human cells to increase production of protective mucus coating of the intestine which alleviates disease symptoms.

3. Create novel diagnostic bacteria, which are administered orally, measure nitrate concentrations while transiting the gut, and then are collected in fecal matter. Expression of reporter protein in response to nitrate can then be measured to discover the nitrate concentration within the patient's gut, allowing for diagnosis of a wide range of diseases such as diabetes, IBS, or arthritis.

4. Create novel bacteria, which can live in plant roots or soil and detect nitrate, a fertilizer component, and when there is a lack of nitrate, supply the plant by producing additional nitrate.

There are (at least) two novel features of this invention.

The most prominent source of novelty is the chimeric NarL/YdfI protein, which is a novel synthetic fusion of domains from two natural proteins. This protein is a new, never produced before, molecule with completely novel signally properties enabling nitrate sensing in B. subtilis.

The second novel component of this invention is the expression of the natural NarX protein in conjunction with the previously mentioned chimeric NarL/YdfI protein in B. subtilis. This NarX protein, and in fact, the whole family of proteins, have not been previously transported from a gram negative bacteria such as E. coli to a gram positive bacteria such as B. subtilis.

There are already documented examples (DeAngelis, 2005) of nitrate-sensing bacteria. However, we are the first to isolate the nitrate-sensing pathway from a range of competing signals such as oxygen and nitrite sensing while still maintaining extremely high change in response to nitrate. This was accomplished by moving the signaling pathway from its natural bacterial host E. coli to the probiotic host B. subtilis using newly designed signaling proteins.

The first step in creating this invention was to bioinformatically align protein sequence of the NarL signaling protein with those of similar proteins from B. subtilis. This allowed us to select the YdfI (36% identity) as the best target protein for a fusion. We then used the alignment to determine an ideal split point containing the first half of NarL and the second half of YdfI. These were identified by selecting the boundaries of the unstructured linker regions between the α5 and α7 domains.

We subsequently used DNA manipulation technique to create a series of DNA sequences which allowed use to produce this protein and several others in B. subtilis. B. subtilis containing this engineered DNA was then grown in our laboratory and the production of a fluorescent protein in response to nitrate was measured. This allowed us to determine the degree of nitrate sensing the engineered bacteria were capable of.

The original nitrate sensor was only sensitive to nitrate in a narrow range of nitrate concentrations. However, there exist several protein and DNA engineering techniques that enable varying the range of sensitivity of this class of proteins.

This approach could be used to engineer sensors for other chemicals whose sensing proteins are homologous to the NarX/NarL protein pair. The most likely successful candidate would be the NarQ/NarP nitrite sensing system.

There are a great variety of reporter genes that can be used herein, and GFP is only one convenient reporter. The amount or activity of the reporter protein produced is taken as a proxy for the cellular response to the target. Importantly, the reporter gene by definition is NOT the wild type downstream target gene, but is artificially coupled to the TCS to provide a more convenient readout.

Ideal reporter proteins are easy to detect and quantify (preferably noninvasively), highly sensitive and, ideally, not present in the native organism. They can be set up to detect either gene activated or deactivation. Several currently popular reporter proteins and their characteristics are listed in TABLE 1.

TABLE 1
Common spectroscopically active reporter proteins and their detection

| | Reporter | | | | | |
|---|---|---|---|---|---|---|
| Reporter protein | genes | Origin | Substrate | Detection method | Comments | Refs |
| Bacterial luciferase | luxAB* or | Bioluminescent | O₂, FMNH₂ and | Bioluminescence | Requires O₂; aldehyde | 94, 95 |
| | luxCDABE | bacteria* | long-chain aldehydes | | addition is required if | |
| | | | | | only luxAB is used | |
| Firefly luciferase | lucFF | Firefly (Photinus pyralis) | O₂, ATP and luciferin | Bioluminescence | Requires O₂ | 96 |
| Click beetle | lucGR | Click beetle (Pyrophorus | O₂, ATP and pholasin | Bioluminescence | Requires O₂ | 97 |
| luciferase | | plagiophthalamus) | | | | |
| Renilla luciferase | Rluc | Renilla reniformis | Coelenterazine and Ca²⁺ | Bioluminescence | Requires O₂ | 98 |
| β-Galactosidase | lacZ | Escherichia coli | Galactopyranosides^{‡} | Chemiluminescence, | External substrate | 1 |
| | | | | colorimetry, | addition (may require | |
| | | | | electrochemistry and | cell permeabilization) | |
| | | | | fluorescence | | |
| Fluorescent proteins | gfp. etc. | Aequorea victoria and | N/A | Fluorescence | O₂ is required for | 99-101 |
| | | additional marine | | | maturation; different | |
| | | invertebrates | | | colour varieties exist | |
| Spheroidene | crtA | Rhodovulum | Spheroidene | Colorimetry | None | 102 |
| monooxygenase | | sulfidophilum | | | | |
| Infrared fluorescent | Various | Bacteriophytochrome | N/A | Fluorescence | None | 103 |
| proteins | | family | | | | |
| FMN-based | Various | Engineered from | None | Fluorescence | Functional in both oxic | 104 |
| fluorescent proteins | | Bacillus subtilis and | | | and anoxic conditions; | |
| | | Pseudomonas putida | | | requires endogenous | |
| | | | | | FMN | |

N/A, not applicable.
*Most commonly used species inciude Aliivibrio fischeri (also known as Vibrio fischeri), Vibrio harveyi and Photorhabdus luminescens.
^{‡}For example, O-nitrophenyl-β-D-galactoside (ONPG), 5-bromo-4-chloro-3-indolyl-β-D-galactopyranoside (X-gal),4-methylumbelliferyl-β-D-galoctopyranoside, 4-aminophenyl-β-D-galactopyranside and D-luciferin-O-β-galactopyranoside.

Using the amount of reporter gene as a readout, and using standard high throughput screening methods, such as fluorimetry or flow-cytometry, we can screen potential nitrate sensing TCSs for activity using standard, high throughput laboratory assays. In this way, we can expand the range of nitrate sensor genes that can be employed herein.

Initial experiments proceeded in E. coli and B. subtillus for convenience, but the addition of genes to bacteria is of nearly universal applicability, so it will be possible to use a wide variety of organisms with the selection of suitable vectors for same. Furthermore, a number of databases include vector information and/or a repository of vectors. See e.g., Addgene.org, which provides both a repository and a searchable database allowing vectors to be easily located and obtained from colleagues. See also Plasmid Information Database (PlasmID) and DNASU having over 191,000 plasmids. A collection of cloning vectors of E. coli is also kept at the National Institute of Genetics as a resource for the biological research community. Furthermore, vectors (including particular ORFS therein) are usually available from colleagues.

Once an exemplary sequence is obtained, e.g., in E. coli, which is completely sequenced and which is the workhorse of genetic engineering and bioproduction, many additional examples proteins of similar activity can be identified by BLAST search or database search. The OMIN database is also a good resource for searching human proteins and has links to the sequences. Further, every protein record is linked to a gene record, making it easy to design genome insertion vectors. Many of the needed sequences are already available in vectors, and can often be obtained from cell depositories or from the researchers who cloned them. But, if necessary, new clones can be prepared based on available sequence information using gene synthesis or PCR techniques. Thus, it should be easily possible to obtain all of the needed sequences.

Understanding the inherent degeneracy of the genetic code allows one of ordinary skill in the art to design multiple sequences that encode the same amino acid sequence. NCBI® provides codon usage databases for optimizing DNA sequences for protein expression in various species. Using such databases, a gene or cDNA may be “optimized” for expression in probiotic strains, mice, humans, or other species using the codon bias for the species in which the gene will be expressed.

In calculating “% identity” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence).

Alignments are performed using BLAST homology alignment as described by Tatusova T A & Madden T L (1999) FEMS Microbiol. Lett. 174:247-250. The default parameters were used, except the filters were turned OFF. As of Jan. 1, 2001 the default parameters were as follows: BLASTN or BLASTP as appropriate; Matrix=none for BLASTN, BLOSUM62 for BLASTP; G Cost to open gap default=5 for nucleotides, 11 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W word size [Integer] default=11 for nucleotides, 3 for proteins; y Dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at NCBI™ (ncbi.nlm.nih.gov/BLAST/). “Positives” includes conservative amino acid changes in addition to identities.

As used herein, a “two component system” or “two component sensor system” or “TCS” is understood to be a two protein system including a sensor kinase and a response regulator, wherein the sensor kinase when bound to its cognate ligand, activates the response regulator which then activates the expression of relevant downstream proteins.

As used herein, a “sensor kinase” or “SK” is a protein understood to have a ligand binding domain (“LBD”) operably coupled to a “kinase domain” (“KD”), such that when the LBD binds its cognate ligand (in this application nitrate), the kinase is activated.

“Cognate” refers to two components systems that function together, such that a SK will bind to its cognate RR and activate it. The SK and RR are thus cognate, meaning they function together, or are related or connected functionally.

As used herein, a “response regulator” or “RR” typically has a “receiver” or “REC” domain that is activated by the active kinase of the cognate TCS. Typically the REC domain is operably coupled to a “DNA binding domain” or “DBD,” which thus can bind to and turn on relevant downstream protein expression, such as a report gene. If the native downstream cognate promoters are not known, or are insufficiently active, the DBD domain can be replaced with a more suitable one, thus “rewiring” the RR.

As used herein, a “heterologous DBD” means a DBD that comes from another protein, not the response regulator that the REC domain comes from. Typically, the DBD then binds to the DNA it is targeted to, which is itself coupled to a reporter gene that can easily be detected.

The term “output promoter” means a promoter that is responsive to the TCS used herein. It is operably coupled to a “reporter gene” or a therapeutic protein gene, meaning that the output promoter controls the expression of said gene, typically by binding the DBD of the RR.

A “reporter gene” is an easily monitored gene that is heterologous to said output promoter (thus the normal downstream target is by definition excluded), and preferably is not present in the host species. Fluorescent proteins make excellent reporters.

As used herein, reference to cells, bacteria, microbes, microorganisms and like is understood to include progeny thereof having the same genetic modifications. It is also understood that all progeny may not be precisely identical in DNA content, due to deliberate or inadvertent mutations that have been added to the parent. Mutant progeny that have the same function or biological activity as screened for in the originally transformed cell are included. Where distinct designations are intended, it will be clear from the context.

The terms “operably associated” or “operably linked,” as used herein, refer to functionally coupled nucleic acid sequences.

As used herein “recombinant” or “engineered” is relating to, derived from, or containing genetically engineered material. In other words, the genome was intentionally manipulated in some way by the hand-of-man.

“Reduced activity” or “inactivation” is defined herein to be at least a 75% reduction in protein activity, as compared with an appropriate control species.

Preferably, at least 80, 85, 90, 95% reduction in activity is attained, and in the most preferred embodiment, the activity is eliminated (100%, aka a “knock-out” or “null” mutants which produce undetectable levels of activity). Proteins can be inactivated with inhibitors, by mutation, or by suppression of expression or translation, and the like. Use of a frame shift mutation, early stop codon, point mutations of critical residues, or deletions or insertions, and the like, can completely inactivate (100%) gene product by completely preventing transcription and/or translation of active protein.

“Overexpression” or “overexpressed” is defined herein to be at least 150% of protein activity as compared with an appropriate control species, and preferably 200, 500, 1000%) or more, or any expression is a species that otherwise lacks the activity. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like.

The term “endogenous” or “native” means that a gene originated from the species in question, without regard to subspecies or strain, although that gene may be naturally or intentionally mutated, or placed under the control of a promoter that results in overexpression or controlled expression of said gene. Thus, genes from Clostridia would not be endogenous to Escherichia, but a plasmid expressing a gene from E. coli would be considered to be endogenous to any genus of Escherichia, even though it may now be overexpressed. By contrast, “wild type” means the natural functional gene/protein as it exists in nature.

The invention includes any one or more of the following embodiment(s) in any combination(s) thereof:
A genetically engineered bacteria, said bacteria overexpressing:
a two component sensor system (TCS) comprising:
   a nitrate-sensing sensor kinase (SK) gene comprising a ligand binding domain operably coupled to a kinase domain, and,
a cognate response regulator (RR) gene comprising a receiver domain operably coupled to an DNA binding domain (DBD),
an output promoter that binds said DBD that is operably coupled to a heterologous reporter gene.
A genetically engineered bacteria, said bacteria expressing:
a heterologous two component sensor system (TCS) comprising:
   a nitrate-sensing sensor kinase (SK) gene comprising a ligand binding domain operably coupled to a kinase domain, and,
a cognate response regulator (RR) gene comprising a receiver domain operably coupled to an DNA binding domain (DBD),
an output promoter that binds said DBD that is operably coupled to a reporter gene.
A genetically engineered bacteria, said bacteria expressing:
   a two component sensor system (TCS) comprising:
   a nitrate-sensing sensor kinase (SK) gene comprising a ligand binding domain operably coupled to a kinase domain; and,
a rewired cognate response regulator (RR) gene comprising a receiver domain operably coupled to a heterologous DNA binding domain (DBD) that is operable in said bacteria;
a reporter gene comprising an output promoter that is responsive to said DBD that is operably coupled to an open reading frame encoding a reporter protein.
A genetically engineered bacteria, said bacteria expressing:
   a heterologous two component sensor system (TCS) comprising:
   a nitrate-sensing sensor kinase (SK) gene comprising a ligand binding domain operably coupled to a kinase domain; and,
a rewired cognate response regulator (RR) gene comprising a receiver domain operably coupled to a heterologous DNA binding domain (DBD) that is operable in said bacteria;
a reporter gene comprising an output promoter that is responsive to said DBD that is operably coupled to an open reading frame encoding a reporter protein.
A bacteria as herein described, which is probiotic for use in humans. The bacteria might also be probiotic for use in other species, e.g., companion animals, as appropriate for the species of patient being treated.
A bacteria as herein described, wherein said SK gene or said RR gene or both genes are encoded on an expression vector, which can be inducible or constitutive.
A bacteria as herein described, wherein said SK gene or said RR gene or both genes integrated into a genome of said bacteria.
A bacteria as herein described, wherein said SK gene and said RR gene are encoded in a single operon.
A bacteria as herein described, wherein said reporter gene is encoded on a plasmid or other expression vector.
A bacteria as herein described, wherein said reporter gene is integrated into a genome of said bacteria.
A bacteria as herein described, comprising SEQ ID NO. 1 and an amino terminal portion of SEQ ID NO. 3 operably fused to a carboxy terminal portion of SEQ ID NO 5 containing a DNA binding site. SEQ ID. NO. 6-9 could also be used. Homologs of same are also possible. A bacteria as herein described, wherein said reporter gene encodes a fluorescent protein, such as green fluorescent protein, red fluorescent protein, far red fluorescent protein, blue fluorescent protein, orange fluorescent protein, yellow fluorescent protein, mCHERRY, mORANGE, mCITRINE, VENUS, YPET, EMERALD, or CERULEAN.
A method of screening for a gut bacteria that produces nitrate, comprising:
i) applying a bacteria as herein described to a gut or a gut sample; and,
ii) measuring activity of said reporter gene, wherein activation of said reporter gene indicates that said gut or gut sample harbors a gut bacteria that produces nitrate.
A method of screening for a nitrate-generating bacteria, comprising:
i) applying a test sample containing test bacteria to a bacteria as herein described; and, ii) measuring activity of said reporter gene, wherein activation of said reporter gene at a level greater than a normal level indicates that said test sample harbors an excess of a nitrate-generating bacteria.
A method of detecting nitrate, comprising: i) combining a test sample with the bacteria described; and, ii) measuring activity of said reporter gene, wherein expression of said reporter gene correlates with an amount of nitrate in said sample.
A method of detecting nitrate, comprising: i) combining a test sample with the bacteria herein described; and, ii) measuring expression of said reporter gene, wherein a change in a level of expression of said reporter gene as compared to a control sample lacking nitrate indicates that said test sample contains nitrate.
A method of detecting nitrate in soil, comprising: i) combining a test sample of soil with the bacteria herein described; ii) measuring expression of said reporter gene; and iii) correlating a measured level of reporter gene expression with a level of nitrate in said test sample of soil using a standard curve.
A method of detecting excess nitrate levels in a patient, comprising i) administering the bacteria of claim 5-15 to a patient, ii) collecting a stool sample from said patient; iii) measuring expression of said reporter gene in said stool sample, wherein a change in level of expression of said reporter gene over a normal level in a normal patient indicates that said patient has excess nitrate.
A method of measuring nitrate levels in a patient, comprising:
a) combining a gut sample with a nitrate reporter bacteria comprising:
i) a nitrate-sensing sensor kinase (SK) gene encoding an SK protein comprising a ligand binding domain that binds nitrate and activates a kinase domain,
ii) a cognate RR gene encoding an RR protein comprising a receiver domain operably coupled to an DNA binding domain (DBD), wherein said cognate RR protein is activated by said activated kinase domain phosphorylating said receiver domain, and
iii) a reporter gene comprising a DNA binding site that binds said DBD of said cognate activated RR protein operably coupled to an open reading frame encoding a reporter protein;
b) measuring expression of said reporter gene; and,
c) correlating a measured level of reporter gene expression with a level of nitrate using a standard curve.
A method as herein described, wherein said bacteria is a probiotic bacteria and said combining step a is by administering said bacteria to said patient.
A method as herein described, wherein said combining step a is by collecting a stool sample from said patient and combining said stool sample with said bacteria.
A genetically engineered probiotic bacteria, said probiotic bacteria overexpressing:
a heterologous nitrate sensor system comprising:
i) a nitrate-sensing sensor kinase (SK) comprising a ligand binding domain operably coupled to a kinase domain; and,
ii) a cognate response regulator (RR) comprising a receiver domain operably coupled to an DNA binding domain (DBD);
a DNA binding site that binds said DBD that is operably coupled to either a reporter gene or a therapeutic protein gene.
A bacteria as herein described, wherein said RR GENE is rewired such that said receiver domain is operably coupled to a heterologous DBD from another response regulator.
A treatment method, comprising administering the probiotic bacteria described herein to a patient having excess nitrate, wherein said DBD is operably coupled to a therapeutic protein.
A fusion protein comprising the amino terminus of NarL operably fused to the DNA binding site domain of YdfI.
The fusion protein comprising an amino portion of SEQ ID NO 3 fused to a carboxy portion of SEQ ID NO. 5, or comprising SEQ ID NO. 6-9, or homologs of any of same.
A bacteria comprising an expression vector encoding the fusion protein herein described.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, background mutations that do not effect the invention, and the like.

The following abbreviations are used herein:

| ABBREVIATION | TERM |
|---|---|
| ATP | Adenosine triphosphate |
| CSE | C minimal medium with sodium succinate |
| | (6 g/l) and potassium glutamate (8 g/l), |
| | a well known Bacillus medium |
| DBD | DNA binding domain |
| EAEC | enteroaggregative E. coli |
| EHEC | enterohemorrhagic E. coli |
| EIEC | enteroinvasive E. coli |
| EPEC | enteropathogenic E. coli |
| ETEC | enterotoxigenic E. coli |
| GFP | Green fluorescent protein |
| HK | Histidine kinases |
| HPT | Histidine phosphotransferase |
| IPTG | Isopropyl β-D-1-thiogalactopyranoside |
| KD | Kinase domain |
| LBD | Ligand binding domain |
| PBS | Phosphate buffered saline |
| REC | Receiver domain |
| RR | Response regulator |
| SK | Sensor kinase |
| TCS | Two component sensor system including |
| | a KD and an RR |

### BRIEF DESCRIPTION OF FIGURES

FIG. 1. Two component sensor systems.

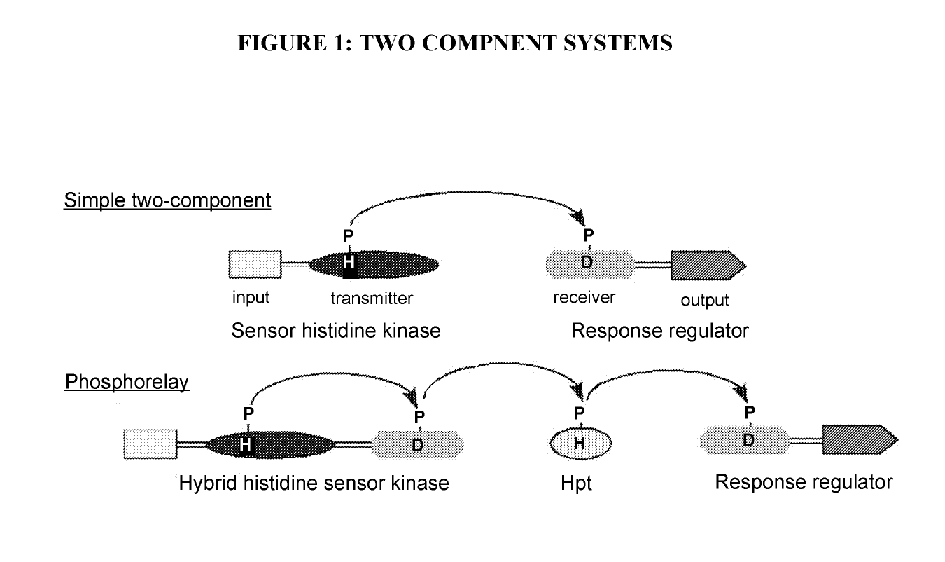

FIG. 2. Overview of the human colonic microflora. Bacterial genera were classified as health positive, health negative, or health neutral. Bacterial enumeration was by selective media. Ps=Pseudomonas.

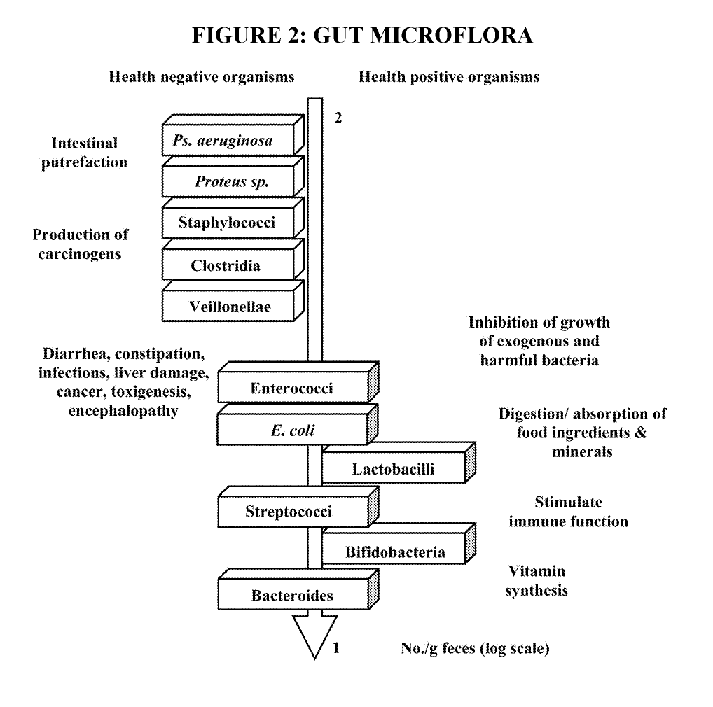

FIG. 3. Dose response of the described NarX and NarL/YdfI nitrate sensor in B. subtilis to NaNO₃.

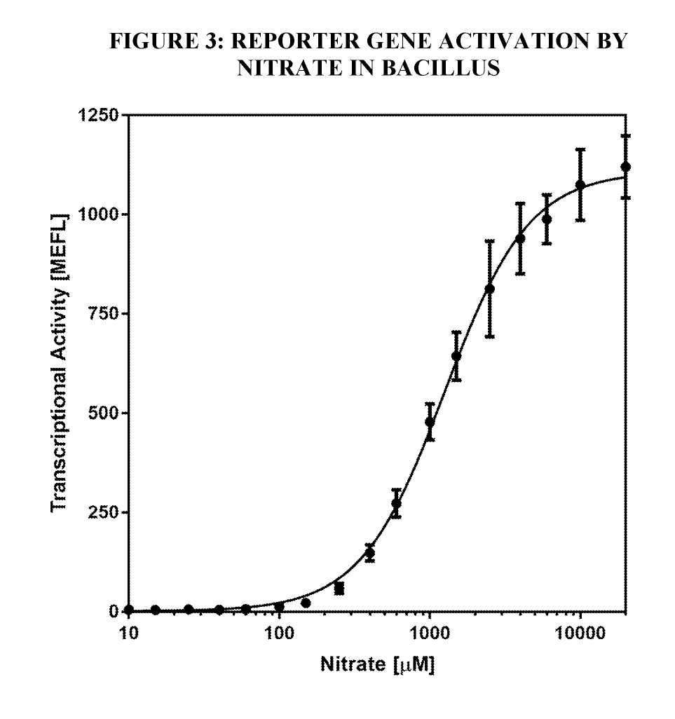

FIG. 4. Demonstration of the use of the B. subtilis nitrate sensor to detect both nitrate and fertilizer in a soil sample. Dose response curves of soil with increasing concentrations of NaNO₃ and fertilizer are shown.

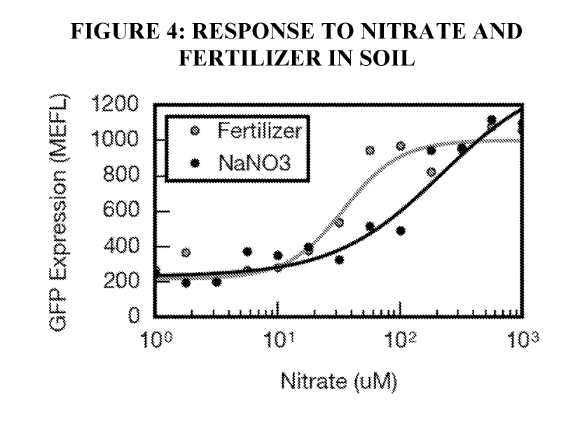

FIG. 5. Dose response of the described NarX and NarL/YdfI nitrate sensor in E. coli to NaNO₃. An additional inactivated NarL-YdfI D59E mutant (lacking the needed aspartate residue for activation) is shown as a negative control to demonstrate specificity of the response to the described pathway.

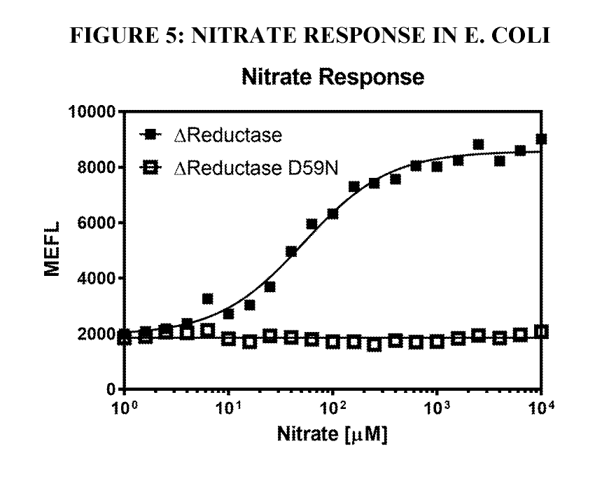

FIG. 6. Preliminary data demonstrating the use of the E. coli nitrate sensor being used in vivo to determine the presence of the dextran sodium sulfate (DSS) inflammation disease model in mice.

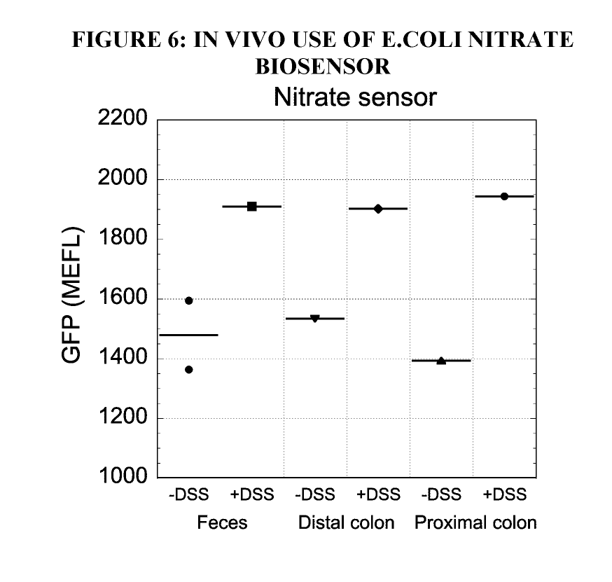

### DETAILED DESCRIPTION

In our proof of concept work, we used a NarX SK and a rewired NarL RR with a DBD domain from YdfI and a GFP reporter. These sequence are publically available, and are discussed in more detail below.

NarX: Acts as a sensor kinase (SK) for nitrate/nitrite and transduces signal of nitrate availability to the NarL protein and of both nitrate/nitrite to the NarP protein. NarX probably activates NarL and NarP by phosphorylation in the presence of nitrate. NarX also plays a negative role in controlling NarL activity, probably through dephosphorylation in the absence of nitrate.

narX from E. coli [1](SEQ ID NO. 1):
MLKRCLSPLT LVNQVALIVL LSTAIGLAGM AVSGWLVQGV
QGSAHAINKA GSLRMQSYRL LAAVPLSEKD KPLIKEMEQT
AFSAELTRAA ERDGQLAQLQ GLQDYWRNEL IPALMRAQNR
ETVSADVSQF VAGLDQLVSG FDRTTEMRIE TVVLVHRVMA
VFMALLLVFT IIWLRARLLQ PWRQLLAMAS AVSHRDFTQR
ANISGRNEMA MLGTALNNMS AELAESYAVL EQRVQEKTAG
LEHKNQILSF LWQANRRLHS RAPLCERLSP VLNGLQNLTL
LRDIELRVYD TDDEENHQEF TCQPDMTCDD KGCQLCPRGV
LPVGDRGTTL KWRLADSHTQ YGILLATLPQ GRHLSHDQQQ
LVDTLVEQLT ATLALDRHQE RQQQLIVMEE RATIARELHD
SIAQSLSCMK MQVSCLQMQG DALPESSREL LSQIRNELNA
SWAQLRELLT TFRLQLTEPG LRPALEASCE EYSAKFGFPV
KLDYQLPPRL VPSHQAIHLL QIAREALSNA LKHSQASEVV
VTVAQNDNQV KLTVQDNGCG VPENAIRSNH YGMIIMRDRA
QSLRGDCRVR RRESGGTEVV VTFIPEKTFT DVQGDTHE
NarQ: another nitrate detector from Shewenella
oneidensis. SEQ ID NO. 2:
MKRGSLTSKI LGLMLVLILL SSSLAIFAII NLSYSLGDAK
AINASGSLRM QSYRLMFYAN SGSEAAQEKI TEFENTLHSE
ALHPSKSWLS PKKIAAQYQL VIDKWLVMKY YIEQENSRDY
AASLKDFVDT IDLLVLEMEH HAAFKLRLLA ASQIFGLGLM
LSIAFLAVRF TKRKVVVPLQ QLMESANTIS KGNFEIEMPE
TEYIELTALT DALQKTAREL ATLYGNLESQ VAEKTLALTR
ANNELAFLYD TLLTLNAKKL DYKALKAALN QLKDYESIDY
LRLIIQYPEQ ELEMIEANGG WPESADNSTR FPLQFEQANL
GYLELISAQD INTPLFKNFA IMLTRSIVIH NATEQRQQLA
LMEERGVIAR ELHDSLGQVL SFLKIQISLL RKNLDHSCRS
PAVEVQLTEI NEGVSTAYVQ LRELLSTFRL TIKEPNLKNA
MEAMLEQLRA NTDIKIHLDY KLSPQWLEAK QHIHILQITR
EATLNAIKHA NASHINIRCY KDDRGMVNIS VSDNGVGIGH
IKERDQHFGI GIMHERASKL DGEVVFSSND THTNSTATTE
QRHQENPDSP LESHNTSNLS QGTIVTLIFP SQQEPTHG

Other nitrate SK homologs that can be used include WP 042949651 from Salmonella (86% amino acid identity to NarX); WP_042949651 from Citrobacter (86%); WP_045142747.1 from Enterobacter (94%); and WP_059179795.1 from Lelliottia (81%). As can be seen, the degree of homology is quite high, indicating a high likelihood of having the same functionality.

Additional proteins that can substitute herein can be identified by homology search, and functionality can be confirmed as described herein. These are available by BLAST search of the above sequences at GenBank. Additionally, UniProt and other such databases have links to a large number of variants in the same and different species.

NarL: This response regulator (RR) protein activates the expression of the nitrate reductase (narGHJI) and formate dehydrogenase-N(fdnGHI) operons and represses the transcription of the fumarate reductase (frdABCD) operon in response to a nitrate/nitrite induction signal transmitted by either the NarX or NarQ proteins. The DNA binding element is 173-192 (underlined).

NarL from E. coli. (SEQ ID NO. 3):
MSNQEPATIL LIDDHPMLRT GVKQLISMAP DITVVGEASN
GEQGIELAES LDPDLILLDL NMPGMNGLET LDKLREKSLS
GRIVVFSVSN HEEDVVTALK RGADGYLLKD MEPEDLLKAL
HQAAAGEMVL SEALTPVLAA SLRANRATTE RDVNQLTPRE
RDILKLIAQG LPNKMIARRL DITESTVKVH VKHMLKKMKL
KSRVEAAVWV HQERIF
NarP: another Shewenella frigidmarina RR believed
to respond to NarX and/or NarQ. SEQ ID NO. 4:
MGKPYSVLVV DDHPLLRRGI CQLITSDGDF SLFGETGTGL
EALTAVAEDE PDIILLDLNM KGMSGLDTLN AMRQEGVTAR
IVILTVSDAK QDVVRLLRAG ADGYLLKDTE PDLLLEQLKK
AMLGHRVISD EVEAYLYELK NTIDDNSWIE NLTPRELQIL
QELAEGKSNR MIAEDLHISE GTVKVHVKNL LRKANAKSRT
EMAVRYLNN

Additional nitrate RR homologs that can be used herein include WP_000070489.1 from Shigella (99%); WP_045443652.1 from Citrobacter (98%); WP_061496301.1 from Enterobacter (97%); WP_003856701.1 from Proteobacter (96%); WP_032641051.1 from Enterobacter (96%); WP_001064598.1 from Salmonella (96%); WP 020803248.1 from Kleibsella (94%); WP 032611305.1 from Leclercia (96%); and WP_035895589.1 from Kluyvera (95%).

YdfI: An RR member of the two-component regulatory system YdfH/YdfI. Regulates the transcription of ydfJ by binding to its promoter region. The DNA binding subsequence is aa 166-186 (underlined).

YdfI from Bacillus subtilis. (SEQ ID NO. 5):
MNKVLIVDDH LVVREGLKLL IETNDQYTII GEAENGKVAV
RLADELEPDI ILMDLYMPEM SGLEAIKQIK EKHDTPIIIL
TTYNEDHLMI EGIELGAKGY LLKDTSSETL FHTMDAAIRG
NVLLQPDILK RLQEIQFERM KKQRNETQLT EKEVIVLKAI
AKGLKSKAIA FDLGVSERTV KSRLTSIYNK LGANSRTEAV
TIAMQKGILT IDN
Exemplary NarL-YdfI fusion protein (SEQ ID NO. 6),
the NarL split at aa 170 (Ydfl underlined):
MSNQEPATIL LIDDHPMLRT GVKQLISMAP DITVVGEASN
GEQGIELAES LDPDLILLDL NMPGMNGLET LDKLREKSLS
GRIVVFSVSN HEEDVVTALK RGADGYLLKD MEPEDLLKAL
HQAAAGEMVL SEALTPVLAA SLRANRATTE RDVNQLTPRE
RDILKLIAQG AKGLKSKAIA FDLGVSERTV KSRLTSIYNK
LGANSRTEAV TIAMQKGILT IDN

As of yet, there are no examples of this technique succeeding with a DBD from a non-TCS, but it is possible (albeit unlikely) if the domain structure were such as to be activatable by an active REC domain. However, there are a large number (>10,000 TCS) of proteins available from which to choose, so this limitation is very modest. A homologous DBD from the native RR is predicted to give the best chance of success (>30%, >35%, >40%, or higher), but we have used non-homologous domains too.

Obviously, the heterologous DBD domain that is rewired to the RR should be functional in the bacterial species in which the nitrate sensor will be hosted. In making the change from disparate species, it may be necessary to select a DBD domain from the host species or a closely related species to ensure operability. In this way, we were able to move a heterologous TCS system from a gram negative (E. coli) to a gram positive (B. subtillus) species.

The exact fusion point of the two domains can vary somewhat, provided that the DNA binding subsequence (underlined) of NarL (or a homolog) is replaced with that of YdfI or another suitable DBD from a heterologous RR. By switching the DBD domains, we are able to transport the nitrate sensor system of E. coli into the probiotic strain of B. Subtilus.

Other potential DBDs that can be used herein include LiaR (UniProt 032197) at the linker region in the 20 amino acids surrounding the K120 residue and UhpA (P0AGA6) at the linker region in the 20 amino acids surrounding the T123 residue.

We have also constructed three other chimera RR proteins herein:

NarL131-Ydfl (SEQ. ID NO. 7) which is split at
the 131^{st} amino acid of NarL:
MSNQEPATIL LIDDHPMLRT GVKQLISMAP DITVVGEASN
GEQGIELAES LDPDLILLDL NMPGMNGLET LDKLREKSLS
GRIVVFSVSN HEEDVVTALK RGADGYLLKD MEPEDLLKAL
HQAAAGEMVL SPDILKRLQE IQFERMKKQR NETQLTEKEV
IVLKAIAKGL KSKAIAFDLG VSERTVKSRL TSIYNKLGAN
SRTEAVTIAM QKGILTIDN

The other two chimeric proteins (SEQ. ID NO. 8 and 9) are split at the nearby amino acids NarL142 and NarL154 and have a similar but slightly decreased functionality.

NarL142-Ydfl (SEQ. ID NO. 8):
MSNQEPATIL LIDDHPMLRT GVKQLISMAP DITVVGEASN
GEQGIELAES LDPDLILLDL NMPGMNGLET LDKLREKSLS
GRIVVFSVSN HEEDVVTALK RGADGYLLKD MEPEDLLKAL
HQAAAGEMVL SEALTPVLAA SLQFERMKKQ RNETQLTEKE
VIVLKAIAKG LKSKAIAFDL GVSERTVKSR LTSIYNKLGA
NSRTEAVTIA MQKGILTIDN
NarL154-Ydfl (SEQ. ID NO. 9):
MSNQEPATIL LIDDHPMLRT GVKQLISMAP DITVVGEASN
GEQGIELAES LDPDLILLDL NMPGMNGLET LDKLREKSLS
GRIVVFSVSN HEEDVVTALK RGADGYLLKD MEPEDLLKAL
HQAAAGEMVL SEALTPVLAA SLRANRATTE RDVNQLTEKE
VIVLKAIAKG LKSKAIAFDL GVSERTVKSR LTSIYNKLGA
NSRTEAVTIA MQKGILTIDN

For proof of concept experiments to characterize the nitrate sensor in B. subtilis, the sensor kinase NarX was expressed under the IPTG inducible Phyper_spank promoter in the AmyE locus and the NarL-YdfI gene was expressed from the xylose inducible PxylA promoter at the LacA locus.

Growth/assay protocol for in vitro B. subtilis experiments:
Overnight pre-culture (13 hours) in CSE 0.5% glycerol
Dilute overnight culture in CSE 0.5% glycerol with optimal IPTG and Xylose induction levels
Grow 90-150 minutes
Dilute to OD600=0.001 in CSE 0.5% glycerol with optimal IPTG and Xylose induction levels
Grow shaking at 37° C. until cultures reach OD600=0.1 . . . 0.3
Put on ice, measure fluorescence by flow cytometry

Growth/assay protocol for measuring nitrate in soil:
Overnight pre-culture (13 hours) in CSE 0.5% glycerol
Dilute overnight culture in 1:100 CSE 0.5% glycerol with optimal IPTG and Xylose induction levels
Grow for until OD600=0.1
Add appropriate amounts of either sodium nitrate or commercial fertilizer to the soil
Add 250 μL of culture to 0.1 g of soil and mix
Grow standing at 37° C. for 2 hours
Resuspend in 10× volume PBS
Filter dirt through a Whatman filter
Put on ice, measure fluorescence by flow cytometry

For proof of concept experiments to characterize the use of the engineered nitrate sensor in E. coli, the sensor kinase NarX was expressed under the constitutive promoter J23114 and translated with the ribosome binding site (RBS) apFAB655 on a p15a plasmid backbone. The engineered NarL-YdfI response regulator was expressed under the constitutive promoter Bba_J23115 and translated with the RBS BCD24 on a ColE1 plasmid backbone. Transcription of the various genes can terminated by the B0015, T1, or T0 terminators.

Growth/assay protocol for in vitro E. coli experiments:
Overnight pre-culture (˜13 hours) in LB+Cm/Spec.
Dilute to OD₆₀₀=0.02 in M9+0.4% glycerol.
Grow 3 hours to OD₆₀₀=˜0.3.
Dilute to OD₆₀₀=0.0001 in M9+0.4% glycerol.
Add nitrate.
Grow shaking at 37° C. ˜6 hours to OD₆₀₀=˜0.3.
Put on ice, measure OD, measure fluorescence by flow cytometry (FL1=800, FL3=850).

Growth Assay protocol for detection of nitrate in inflammation mouse models:
Treat mice DSS for 5 days to simulate inflammatory bowel disease
Administer genetically engineered nitrate sensing bacteria prepared according the in vitro protocol above
At 6 hours collect mouse fecal and organ samples
Process samples via resuspension in PBS and subsequent filtration through a 10 μM filter
Put on ice and measure fluorescence by flow cytometry (FL1=800, FL3=850).

The above described vectors, promoters, terminators and other components of the system are exemplary only, and other components could be used. However, the above assays provided proof of concept and confirmed that the above system is indeed a nitrate two-component nitrate sensor system.

Although four SK/RR gene pairs were exemplified herein, and at least one pair (SEQ ID NO. 1 and 6) was tested in two host species, there are two features that indicates broad applicability of the invention. The first feature is tunability, which is particularly important for sensing nitrate because the biological ranges for levels of nitrate in humans has not been studied much. Because this system is tunable, once that range is known the sensor can be easily tuned to sense and provide output at the needed levels.

The second feature piggybacks on the tunability function but also relies on the fact that the inventors have engineered and characterized a suite of DBD, promoters, and reporters for use in this system (described in 62/157,293). When combined, these features allow the inventors to transfer the system to a broad range of microbial species and strains.

Each of the following is incorporated by reference herein in its entirety for all purposes:
Claesen J. & Fischbach M. A., Synthetic Microbes As Drug Delivery Systems, ACS Synthetic Biology 2015 4 (4), 358-364.
Stewart V., Nitrate- and nitrite-responsive sensors NarX and NarQ of proteobacteria, Biochemical Society Transactions February 2003, 31 (1) 1-10;
DeAngelis, Kristen M., Pingsheng Ji, Mary K. Firestone, and Steven E. Lindow. “Two Novel Bacterial Biosensors for Detection of Nitrate Availability in the Rhizosphere.” Applied and Environmental Microbiology 71, no. 12
62/157,293, IDENTIFYING LIGANDS FROM BACTERIAL SENSORS, May 5, 2015

1) A genetically engineered bacteria, said bacteria overexpressing:
a) a two component sensor system (TCS) comprising:
i) a nitrate-sensing sensor kinase (SK) gene comprising a ligand binding domain operably coupled to a kinase domain, and,
ii) a cognate response regulator (RR) gene comprising a receiver domain operably coupled to an DNA binding domain (DBD),
b) an output promoter that binds said DBD that is operably coupled to a heterologous reporter gene.

2) (canceled)

3) (canceled)

4) (canceled)

5) The bacteria of claim 1, which is gut-adapted for use in humans.

6) The bacteria of claim 5, wherein said SK gene or said RR gene or both genes are encoded on an expression vector, an inducible expression vector, and/or a constitutive expression vector.

7) (canceled)

8) (canceled)

9) The bacteria of claim 1, wherein said SK gene or said RR gene or both genes integrated into a genome of said bacteria.

10) The bacteria of claim 1, wherein said SK gene and said RR gene are encoded in a single operon.

11) The bacteria of claim 1, wherein said reporter gene is encoded on a plasmid.

12) The bacteria of claim 1, wherein said reporter gene is integrated into a genome of said bacteria.

13) The bacteria of claim 1, comprising SEQ ID NO. 1 and an amino terminal portion of SEQ ID NO. 3 operably fused to a carboxy terminal portion of SEQ ID NO 5 containing a DNA binding site.

14) The bacteria of claim 1, wherein said reporter gene encodes a fluorescent protein.

15) The bacteria of claim 1, wherein said reporter gene encodes green fluorescent protein, red fluorescent protein, far red fluorescent protein, blue fluorescent protein, orange fluorescent protein, yellow fluorescent protein, mCHERRY, mORANGE, mCITRINE, VENUS, YPET, EMERALD, or CERULEAN.

16) (canceled)

17) (canceled)

18) (canceled)

19) (canceled)

20) (canceled)

21) (canceled)

22) A method of measuring nitrate levels in a patient, comprising:
a) combining a gut sample with a nitrate reporter bacteria comprising:
i) a nitrate-sensing sensor kinase (SK) gene encoding an SK protein comprising a ligand binding domain that binds nitrate and activates a kinase domain,
ii) a cognate RR gene encoding an RR protein comprising a receiver domain operably coupled to an DNA binding domain (DBD), wherein said cognate RR protein is activated by said activated kinase domain phosphorylating said receiver domain, and
iii) a reporter gene comprising a DNA binding site that binds said DBD of said cognate activated RR protein operably coupled to an open reading frame encoding a reporter protein;
b) measuring expression of said reporter gene; and,
c) correlating a measured level of reporter gene expression with a level of nitrate using a standard curve.

23) The method of claim 22, wherein said bacteria is a gut-adapted bacteria and said combining step is by administering said bacteria to said patient.

24) The method of claim 22, wherein said combining step a) is by collecting a gut or stool sample from said patient and combining said gut or stool sample with said bacteria.

25) (canceled)

26) (canceled)

27) (canceled)

28) A fusion protein comprising the amino terminus of NarL operably fused to the DNA binding site domain of YdfI.

29) The fusion protein of claim 28, comprising an amino portion of SEQ ID NO 3 fused to a carboxy portion of SEQ ID NO. 5.

30) The fusion protein of claim 28, comprising SEQ ID NO 6-9.

31) A bacteria comprising an expression vector encoding the fusion protein of claim 28.


SEQUENCE LISTING (9 sequences)

SEQ ID NO: 1 - protein, 598 aa, Escherichia coli
MLKRCLSPLTLVNQVALIVLLSTAIGLAGMAVSGWLVQGVQGSAHAINKAGSLRMQSYRLLAAVPLSEKDKPLIKEMEQTAFSAELTRAAERDGQLAQLQGLQDYWRNELIPALMRAQNRETVSADVSQFVAGLDQLVSGFDRTTEMRIETVVLVHRVMAVFMALLLVFTIIWLRARLLQPWRQLLAMASAVSHRDFTQRANISGRNEMAMLGTALNNMSAELAESYAVLEQRVQEKTAGLEHKNQILSFLWQANRRLHSRAPLCERLSPVLNGLQNLTLLRDIELRVYDTDDEENHQEFTCQPDMTCDDKGCQLCPRGVLPVGDRGTTLKWRLADSHTQYGILLATLPQGRHLSHDQQQLVDTLVEQLTATLALDRHQERQQQLIVMEERATIARELHDSIAQSLSCMKMQVSCLQMQGDALPESSRELLSQIRNELNASWAQLRELLTTFRLQLTEPGLRPALEASCEEYSAKFGFPVKLDYQLPPRLVPSHQAIHLLQIAREALSNALKHSQASEVVVTVAQNDNQVKLTVQDNGCGVPENAIRSNHYGMIIMRDRAQSLRGDCRVRRRESGGTEVVVTFIPEKTFTDVQGDTHE

SEQ ID NO: 2 - protein, 598 aa, Shewanella oneidensis
MKRGSLTSKILGLMLVLILLSSSLAIFAIINLSYSLGDAKAINASGSLRMQSYRLMFYANSGSEAAQEKITEFENTLHSEALHPSKSWLSPKKIAAQYQLVIDKWLVMKYYIEQENSRDYAASLKDFVDTIDLLVLEMEHHAAFKLRLLAASQIFGLGLMLSIAFLAVRFTKRKVVVPLQQLMESANTISKGNFEIEMPETEYIELTALTDALQKTARELATLYGNLESQVAEKTLALTRANNELAFLYDTLLTLNAKKLDYKALKAALNQLKDYESIDYLRLIIQYPEQELEMIEANGGWPESADNSTRFPLQFEQANLGYLELISAQDINTPLFKNFAIMLTRSIVIHNATEQRQQLALMEERGVIARELHDSLGQVLSFLKIQISLLRKNLDHSCRSPAVEVQLTEINEGVSTAYVQLRELLSTFRLTIKEPNLKNAMEAMLEQLRANTDIKIHLDYKLSPQWLEAKQHIHILQITREATLNAIKHANASHINIRCYKDDRGMVNISVSDNGVGIGHIKERDQHFGIGIMHERASKLDGEVVFSSNDTHTNSTATTEQRHQENPDSPLESHNTSNLSQGTIVTLIFPSQQEPTHG

SEQ ID NO: 3 - protein, 216 aa, Escherichia coli
MSNQEPATILLIDDHPMLRTGVKQLISMAPDITVVGEASNGEQGIELAESLDPDLILLDLNMPGMNGLETLDKLREKSLSGRIVVFSVSNHEEDVVTALKRGADGYLLKDMEPEDLLKALHQAAAGEMVLSEALTPVLAASLRANRATTERDVNQLTPRERDILKLIAQGLPNKMIARRLDITESTVKVHVKHMLKKMKLKSRVEAAVWVHQERIF

SEQ ID NO: 4 - protein, 209 aa, Shewanella frigidimarina
MGKPYSVLVVDDHPLLRRGICQLITSDGDFSLFGETGTGLEALTAVAEDEPDIILLDLNMKGMSGLDTLNAMRQEGVTARIVILTVSDAKQDVVRLLRAGADGYLLKDTEPDLLLEQLKKAMLGHRVISDEVEAYLYELKNTIDDNSWIENLTPRELQILQELAEGKSNRMIAEDLHISEGTVKVHVKNLLRKANAKSRTEMAVRYLNN

SEQ ID NO: 5 - protein, 213 aa, Bacillus subtilis
MNKVLIVDDHLVVREGLKLLIETNDQYTIIGEAENGKVAVRLADELEPDIILMDLYMPEMSGLEAIKQIKEKHDTPIIILTTYNEDHLMIEGIELGAKGYLLKDTSSETLFHTMDAAIRGNVLLQPDILKRLQEIQFERMKKQRNETQLTEKEVIVLKAIAKGLKSKAIAFDLGVSERTVKSRLTSIYNKLGANSRTEAVTIAMQKGILTIDN

SEQ ID NO: 6 - protein, 223 aa, Artificial Sequence
  NarL+YdfI
MSNQEPATILLIDDHPMLRTGVKQLISMAPDITVVGEASNGEQGIELAESLDPDLILLDLNMPGMNGLETLDKLREKSLSGRIVVFSVSNHEEDVVTALKRGADGYLLKDMEPEDLLKALHQAAAGEMVLSEALTPVLAASLRANRATTERDVNQLTPRERDILKLIAQGAKGLKSKAIAFDLGVSERTVKSRLTSIYNKLGANSRTEAVTIAMQKGILTIDN

SEQ ID NO: 7 - protein, 219 aa, Artificial Sequence
  NarL split at 131 plus the DNA binding sequence from YdfI
MSNQEPATILLIDDHPMLRTGVKQLISMAPDITVVGEASNGEQGIELAESLDPDLILLDLNMPGMNGLETLDKLREKSLSGRIVVFSVSNHEEDVVTALKRGADGYLLKDMEPEDLLKALHQAAAGEMVLSPDILKRLQEIQFERMKKQRNETQLTEKEVIVLKAIAKGLKSKAIAFDLGVSERTVKSRLTSIYNKLGANSRTEAVTIAMQKGILTIDN

SEQ ID NO: 8 - protein, 220 aa, Artificial Sequence
  NarL split at a.a. 142 plus the DNA binding sequence from YdfI
MSNQEPATILLIDDHPMLRTGVKQLISMAPDITVVGEASNGEQGIELAESLDPDLILLDLNMPGMNGLETLDKLREKSLSGRIVVFSVSNHEEDVVTALKRGADGYLLKDMEPEDLLKALHQAAAGEMVLSEALTPVLAASLQFERMKKQRNETQLTEKEVIVLKAIAKGLKSKAIAFDLGVSERTVKSRLTSIYNKLGANSRTEAVTIAMQKGILTIDN

SEQ ID NO: 9 - protein, 220 aa, Artificial Sequence
  NarL split at a.a. 154 plust the DNA binding sequence from YdfI
MSNQEPATILLIDDHPMLRTGVKQLISMAPDITVVGEASNGEQGIELAESLDPDLILLDLNMPGMNGLETLDKLREKSLSGRIVVFSVSNHEEDVVTALKRGADGYLLKDMEPEDLLKALHQAAAGEMVLSEALTPVLAASLRANRATTERDVNQLTEKEVIVLKAIAKGLKSKAIAFDLGVSERTVKSRLTSIYNKLGANSRTEAVTIAMQKGILTIDN